(19) United States
(12) Patent Application Publication
(10) Pub. No.: US 2013/0101557 A1
(43) Pub. Date: Apr. 25, 2013

(54) Recombinant Adenovirus Having Anti-Angiogenesis Activity

(71) Applicant: Chae Ok Yun, Seoul (KR)

(72) Inventor: Chae Ok Yun, Seoul (KR)

(73) Assignee: Industry-University Cooperation Foundation Hanyang University, Seoul (KR)

(21) Appl. No.: 13/519,934

(22) PCT Filed: Nov. 9, 2010
(86) PCT No.: PCT/KR10/07864

§ 371 Date: Jan. 11, 2013

(30) Foreign Application Priority Data
Dec. 31, 2009 (KR) 10-2009-0135629

(51) Int. Cl. A61K 35/76; A61P 35/00; A61P 9/10; A61P 27/02; A61P 17/06; A61P 19/02; A61P 17/02; A61P 37/06; A61P 27/06; A61P 9/08; A61P 29/00; A61P 19/08; A61P 1/00; A61P 1/04; A61P 1/16; A61P 13/12; A61P 3/10; A61P 25/00; C12N 7/01

(52) U.S. Cl. 424/93.2; 435/235.1

(57) ABSTRACT

The present disclosure relates to a recombinant adenovirus with improved angiogenesis inhibition activity and a pharmaceutical composition for inhibiting angiogenesis. The recombinant adenovirus includes: (a) an inverted terminal repeat (ITR) nucleotide sequence of an adenovirus; and (b) a nucleotide sequence coding for a chimeric decoy receptor containing (i) an extracellular domain of vascular endothelial growth factor receptor 1 (VEGFR-1) and (ii) an extracellular domain of vascular endothelial growth factor receptor 2 (VEGFR-2). The recombinant adenovirus according the present disclosure which expresses the chimeric decoy receptor inhibits angiogenesis very effectively and can be used for gene therapy for various angiogenesis-related diseases. Particularly, the recombinant adenovirus of the present disclosure has superior oncolytic activity.

### TECHNICAL FIELD

The present disclosure relates to a recombinant adenovirus which expresses a chimeric decoy receptor and has improved angiogenesis inhibition activity, and a pharmaceutical composition for inhibiting angiogenesis including the same.

### BACKGROUND ART

Angiogenesis is the physiological process involving the growth of new blood vessels from pre-existing vessels. This elaborately regulated process begins from the degradation of the extracellular matrix and the basement membrane and is completed through division, differentiation and invasion into nearby stroma of capillary endothelial cells followed by reorganization into a novel functional vascular network¹. For angiogenesis, various kinds of growth factors are necessary, among which vascular endothelial growth factors (VEGF), particularly VEGF-A, have been found to play an important role. 7 human VEGF-A isoforms (VEGF121, VEGF145, VEGF148, VEGF165, VEGF183, VEGF189 and VEGF206) which are produced through alternative splicing consist of 121, 145, 148, 165, 183, 189 and 206 amino acids, respectively. All of the isoforms share the base sequence of VEGF121²⁻⁴.

Inhibited apoptosis of vascular endothelial cells, lymphangiogenesis, immunosuppression, vascular permeability, hematopoietic stem cell survival, etc. are regulated by the binding between VEGF and VEGF receptor⁴⁻⁷.

Solid tumor can grow only up to maximum size of 2-3 mm in the absence of blood vessels. For further growth, angiogenesis mediated by VEGF is essential for supply of oxygen and nutrients. In normal tissue, the vascular network is hierarchically organized with effective blood flow rate and uniform vessel widths through appropriate proportion of inducing factors and inhibiting factors⁵. However, the vascular system observed in tumors shows increased permeability of vessel walls, high internal pressure and abnormally developed blood vessels. Uncontrolled angiogenesis and abnormal vascular network in tumors are caused by the intracellular signals resulting from the binding between VEGF highly expressed by hypoxia and low pH in the tumor and its receptor VEGFR2⁹.

Angiogenesis induced by VEGF plays a crucial role not only in the growth of tumors but also in infiltration and infiltration and metastasis¹⁰. It has been found out that VEGF is overexpressed in various tumors including lung, stomach, renal, bladder, ovarian and uterine cancer and that the prognosis is worse in cancers where the VEGF is highly expressed¹¹. Since increased blood flow through angiogenesis is essential for tumor growth, inhibition of angiogenesis in tumors is a major target for treatment of cancer. At present, angiostatin, endostatin, thrombospondin-1, uPA-fragment, etc. are used as angiogenesis inhibitor and studies are actively carried out on inhibition of tumor growth or metastasis by suppression of VEGF activity or function of VEGFR-1 (Flt-1) or VEGFR-2 (KDR) which are VEGF receptors¹²⁻¹⁶. Treatment of human tumor xenografts in immunodeficient mice with neutralizing antibodies capable of inhibiting binding of VEGF with its receptor or neutralizing antibodies specific for VEGFR-1 or VEGFR-2 induced apoptosis of vascular endothelial cells and resulted in remarkably inhibited tumor growth¹⁷.

The VEGF trap is a soluble decoy VEGF receptor that is constructed by fusing the domains of VEGFR1 and VEGFR2 on the cell surface. It has high affinity for VEGF. Many studies are being carried out on the VEGF trap and many VEGF traps with improved affinity for VEGF-A, VEGF-B and placental growth factor (PGF) have been constructed¹⁸. The antitumor effect of the VEGF trap was verified in pre-clinical tests of various tumor xenograft models¹⁹⁻²¹ and improved tumor growth inhibition could be achieved through combination therapy with a commercially available anticancer agent as compared to when treated only with the VEGF trap or the anticancer agent²². The reason why the VEGF trap exhibits improved antitumor effect over the anti-VEGF monoclonal antibody bevacizumab or the anti-VEGFR2 antibody DC101 is because it has high affinity for all VEGF isoforms and is capable of binding to PGF²³. Accordingly, continued expression of the VEGF trap having strong affinity for VEGF in tumors is expected to result in excellent antitumor effect by significantly reducing the expression of VEGF in the tumors and provide a significant therapeutic effect.

Adenoviruses are highly esteemed as vectors for cancer gene therapy because of superior gene transfer efficiency as well as high titer and easy concentration²⁴⁻²⁵. However, for the adenovirus-based anticancer agent to be used clinically, development of one capable of selectively and effectively killing cancer cells without harming nearby normal tissue is essential. Since mutation of p53 protein or retinoblastoma protein (pRb) is frequent or the pRb-associated signaling pathway is highly impaired in tumor cells, the adenovirus with pRb binding ability lost is actively replicated in tumor cells whereas the replication is inhibited in normal cells due to pRb activity. As a result, the adenovirus can selectively kill cancer cells. In order to enhance the cancer cell-specific replicating ability of the oncolytic adenovirus, the inventors of the present disclosure have constructed the improved oncolytic adenovirus Ad-ΔB7 which can replicate selectively only in p53-inactivated tumor cells and thus can induce cancer cell-specific apoptosis by replacing the amino acid Glu at CR1 of the E1A gene of adenovirus, which is involved in binding with pRb, with Gly and replacing the 7 amino acids (DLTCHEA) at CR2 with Gly (GGGGGGG) and, at the same time, removing the 55-kDa E1B gene that inhibits p53 protein and the 19-kDa E1B that inhibits apoptosis and reported its antitumor effect in and ex vivo²⁶⁻²⁸.

Throughout the specification, a number of publications and patent documents are referred to and cited. The disclosure of the cited publications and patent documents is incorporated herein by reference in its entirety to more clearly describe the state of the related art and the present disclosure.

### DISCLOSURE

### Technical Problem

The inventors of the present disclosure have studied to improve angiogenesis inhibition activity, particularly oncolytic activity, of an adenovirus by inserting an exogenous sequence into the adenoviral genome. As a result, they have found out that when a nucleotide sequence coding for a chimeric decoy receptor of VEGFR is inserted into the adenoviral genome and expressed, the angiogenesis inhibition activity, particularly oncolytic activity, of the adenovirus is improved remarkably.

The present disclosure is directed to providing a recombinant adenovirus which expresses a chimeric decoy receptor and has improved angiogenesis inhibition activity.

The present disclosure is also directed to providing a pharmaceutical composition for inhibiting angiogenesis containing a recombinant adenovirus which expresses a chimeric decoy receptor.

The present disclosure is also directed to providing a method for preventing or treating a disease caused by excessive angiogenesis.

Other features and aspects will be apparent from the following detailed description, drawings and claims.

### Technical Solution

In one general aspect, the present disclosure provides a recombinant adenovirus with improved angiogenesis inhibition activity comprising: (a) an inverted terminal repeat (ITR) nucleotide sequence of an adenovirus; and (b) a nucleotide sequence coding for a chimeric decoy receptor comprising (i) an extracellular domain of vascular endothelial growth factor receptor 1 (VEGFR-1) and (ii) an extracellular domain of vascular endothelial growth factor receptor 2 (VEGFR-2).

The inventors of the present disclosure have studied to improve angiogenesis inhibition activity, particularly oncolytic activity, of an adenovirus by inserting an exogenous sequence into the adenoviral genome. As a result, they have found out that when a nucleotide sequence coding for a chimeric decoy receptor of VEGFR is inserted into the adenoviral genome and expressed, the angiogenesis inhibition activity, particularly oncolytic activity, of the adenovirus is improved remarkably.

Angiogenesis whereby new blood vessels grow from pre-existing vessels plays a crucial role in the growth and metastasis of tumors. For angiogenesis to occur, various kinds of growth factors are necessary, among which vascular endothelial growth factors (VEGF) have been found to play an important role in angiogenesis.

The chimeric decoy receptor comprising the extracellular domain of VEGFR-1 and the extracellular domain of VEGFR-2 included in the adenoviral vector of the present disclosure is a kind of so-called VEGF trap. It has superior affinity for VEGF-A, VEGF-B and placental growth factor (PGF), and inhibits angiogenesis by acting as a decoy receptor for the growth factors.

As used herein, the term “decoy receptor” refers to a receptor that inhibits binding of VEGF-A, VEGF-B or PGF with a normal receptor by binding to them.

As used herein, the term “chimeric decoy receptor” refers to a receptor constructed by binding an extracellular domain derived from VEGFR-1 with an extracellular domain derived from VEGFR-2.

The chimeric decoy receptor used in the present disclosure is a chimeric receptor obtained by combining at least one extracellular domain of the 7 extracellular domains of VEGFR-1 with at least one extracellular domain of the 7 extracellular domains of VEGFR-2.

In an exemplary embodiment of the present disclosure, the chimeric decoy receptor comprises at least one extracellular domain of VEGFR-1 selected from a group consisting of a first extracellular domain, a second extracellular domain, a third extracellular domain, a fourth extracellular domain, a fifth extracellular domain, a sixth extracellular domain and a seventh extracellular domain of VEGFR-1 and at least one extracellular domain of VEGFR-2 selected from a group consisting of a first extracellular domain, a second extracellular domain, a third extracellular domain, a fourth extracellular domain, a fifth extracellular domain, a sixth extracellular domain and a seventh extracellular domain of VEGFR-2.

More specifically, the chimeric decoy receptor may comprise: (i) the first extracellular domain of VEGFR-1 and at least one extracellular domain of VEGFR-2 selected from a group consisting of the second extracellular domain, the third extracellular domain, the fourth extracellular domain, the fifth extracellular domain, the sixth extracellular domain and the seventh extracellular domain of VEGFR-2; (ii) the second extracellular domain of VEGFR-1 and at least one extracellular domain of VEGFR-2 selected from a group consisting of the first extracellular domain, the third extracellular domain, the fourth extracellular domain, the fifth extracellular domain, the sixth extracellular domain and the seventh extracellular domain of VEGFR-2; (iii) the third extracellular domain of VEGFR-1 and at least one extracellular domain of VEGFR-2 selected from a group consisting of the first extracellular domain, the second extracellular domain, the fourth extracellular domain, the fifth extracellular domain, the sixth extracellular domain and the seventh extracellular domain of VEGFR-2; (iv) the fourth extracellular domain of VEGFR-1 and at least one extracellular domain of VEGFR-2 selected from a group consisting of the first extracellular domain, the second extracellular domain, the third extracellular domain, the fifth extracellular domain, the sixth extracellular domain and the seventh extracellular domain of VEGFR-2; or (v) the fifth extracellular domain of VEGFR-1 and at least one extracellular domain of VEGFR-2 selected from a group consisting of the first extracellular domain, the second extracellular domain, the third extracellular domain, the fourth extracellular domain, the sixth extracellular domain and the seventh extracellular domain VEGFR-2.

Alternatively, the chimeric decoy receptor may comprise: (i) the first extracellular domain of VEGFR-2 and at least one extracellular domain of VEGFR-1 selected from a group consisting of the second extracellular domain, the third extracellular domain, the fourth extracellular domain, the fifth extracellular domain, the sixth extracellular domain and the seventh extracellular domain of VEGFR-1; (ii) the second extracellular domain of VEGFR-2 and at least one extracellular domain of VEGFR-1 selected from a group consisting of the first extracellular domain, the third extracellular domain, the fourth extracellular domain, the fifth extracellular domain, the sixth extracellular domain and the seventh extracellular domain of VEGFR-1; (iii) the third extracellular domain of VEGFR-2 and at least one extracellular domain of VEGFR-1 selected from a group consisting of the first extracellular domain, the second extracellular domain, the fourth extracellular domain, the fifth extracellular domain, the sixth extracellular domain and the seventh extracellular domain of VEGFR-1; (iv) the fourth extracellular domain of VEGFR-2 and at least one extracellular domain of VEGFR-1 selected from a group consisting of the first extracellular domain, the second extracellular domain, the third extracellular domain, the fifth extracellular domain, the sixth extracellular domain and the seventh extracellular domain of VEGFR-1; or (v) the fifth extracellular domain of VEGFR-2 and at least one extracellular domain of VEGFR-1 selected from a group consisting of the first extracellular domain, the second extracellular domain, the third extracellular domain, the fourth extracellular domain, the sixth extracellular domain and the seventh extracellular domain of VEGFR-1.

The chimeric decoy receptor used in the present disclosure may comprise specifically 2-4 extracellular domains, most specifically 3 extracellular domains.

More specifically, the chimeric decoy receptor may comprise: (i) the first extracellular domain of VEGFR-2, the second extracellular domain of VEGFR-1 and the third extracellular domain of VEGFR-2; (ii) the second extracellular domain of VEGFR-1, the third extracellular domain of VEGFR-2 and the fourth extracellular domain of VEGFR-2; or (iii) the second extracellular domain of VEGFR-1, the third extracellular domain of VEGFR-2, the fourth extracellular domain of VEGFR-2 and the fifth extracellular domain of VEGFR-2.

More specifically, the chimeric decoy receptor may comprise: (i) the second extracellular domain of VEGFR-1, the third extracellular domain of VEGFR-2 and the fourth extracellular domain of VEGFR-1; or (ii) the second extracellular domain of VEGFR-1, the third extracellular domain of VEGFR-2, the fourth extracellular domain of VEGFR-1 and the fifth extracellular domain of VEGFR-1.

Most specifically, the chimeric decoy receptor used in the present disclosure may comprise the second extracellular domain of VEGFR-1, the third extracellular domain of VEGFR-2 and the fourth extracellular domain of VEGFR-2.

The amino acid sequence and the nucleotide sequence of VEGFR-1 and VEGFR-2 are available from GenBank. For example, the nucleotide sequence and the amino acid sequence of the second extracellular domain of VEGFR-1 are SEQ ID NOS 1 and 2, the nucleotide sequence and the amino acid sequence of the third extracellular domain of VEGFR-2 are SEQ ID NOS 3 and 4, and the nucleotide sequence and the amino acid sequence of the fourth extracellular domain of VEGFR-2 are SEQ ID NOS 5 and 6.

In an exemplary embodiment of the present disclosure, the Fc region of immunoglobulin (Ig) may be fused in the chimeric decoy receptor. More specifically the Fc region of IgG, most specifically the Fc region of human IgG is fused. The Fc region of Ig is fused via the N- or C-terminus, specifically C-terminus, of the chimeric decoy receptor.

Specific exemplary nucleotide sequence and amino acid sequence of the Fc region of Ig are SEQ ID NOS 7 and 8.

The nucleotide sequence coding for the chimeric decoy receptor may be contained in a genome of adenoviruses.

To construct the present gene delivery system, it is preferred that the chimeric decoy receptor-encoding nucleotide sequence is contained in a suitable expression construct. According the expression construct, it is preferred that the chimeric decoy receptor-encoding nucleotide sequence is operatively linked to a promoter. The term “operatively linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. According to the present invention, the promoter linked to the chimeric decoy receptor gene is operable in, preferably, animal, more preferably, mammalian cells, to control transcription of the chimeric decoy receptor gene, including the promoters derived from the genome of mammalian cells or from mammalian viruses, for example, U6 promoter, H1 promoter, CMV (cytomegalovirus) promoter, the adenovirus late promoter, the vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter, human GM-CSF gene promoter, inducible promoter, tumor cell specific promoter (e.g., TERT promoter, PSA promoter, PSMA promoter, CEA promoter, E2F promoter and AFP promoter) and tissue specific promoter (e.g., albumin promoter). Most preferably, the promoter is CMV promoter.

Cancer gene therapy using adenoviruses has been highlighted because the expression of therapeutic genes is not required to maintain over the life span of patients and immune responses to adenoviruses are not problematic. Therefore, the present invention utilizes adenoviral genome backbones for cancer gene therapy.

Adenovirus has been usually employed as a gene delivery system because of its mid-sized genome, ease of manipulation, high titer, wide target-cell range, and high infectivity. Both ends of the viral genome contains 100-200 by ITRs (inverted terminal repeats), which are cis elements necessary for viral DNA replication and packaging. The EI region (EIA and EIB) encodes proteins responsible for the regulation of transcription of the viral genome and a few cellular genes. The expression of the E2 region (E2A and E2B) results in the synthesis of the proteins for viral DNA replication.

A small portion of adenoviral genome is known to be necessary as cis elements (Tooza, J. Molecular biology of DNA Tumor viruses, 2nd ed. Cold Spring Harbor Laboratory, Cold Spring Harbor, N.Y. (1981)), allowing substitution of large pieces of adenoviral DNA with foreign sequences, particularly together with the use of suitable cell lines such as 293. In this context, the recombinant adenovirus comprises the adenoviral ITR sequence as an essential sequence as well as the chimeric decoy receptor gene.

It is preferred that the chimeric decoy receptor gene is inserted into either the deleted EI region (EIA region and/or EIB region, preferably, EIB region) or the deleted E3 region, more preferably, the deleted E3 region. Another foreign sequence (e.g., cytokine genes, immuno-costimulatory factor genes, apoptotic genes and tumor suppressor genes) is additionally inserted into the recombinant adenovirus, preferably into either the deleted EI region (EIA region and/or EIB region, preferably, EIB region) or the deleted E3 region, more preferably, the deleted EI region (EIA region and/or EIB region, preferably, EIB region). Furthermore, the inserted sequences may be incorporated into the deleted E4 region.

In nature, adenovirus can package approximately 105% of the wild-type genome, providing capacity for about extra 2 kb of DNA. In this regard, the foreign sequences described above inserted into adenovirus may be further inserted into adenoviral wild-type genome.

According to a preferred embodiment, the recombinant adenovirus of this invention comprises the inactivated EIB 19 gene, inactivated EIB 55 gene or inactivated EIB 19/E1B 55 gene. The term “inactivation” in conjunction with genes used herein refers to conditions to render transcription and/or translation of genes to occur non-functionally, thereby the correct function of proteins encoded genes cannot be elicited. For example, the inactivated EIB 19 gene is a gene incapable of producing the functional EIB 19 kDa protein by mutation (substitution, addition, and partial and whole deletion). The defect EIB 19 gives rise to the increase in apoptotic incidence and the defect EIB 55 makes a recombinant adenovirus tumor-specific (see Korean Pat. Appln. No. 2002-23760). The term used herein “deletion” with reference to viral genome encompasses whole deletion and partial deletion as well.

According to a preferred embodiment, the recombinant adenovirus of the present invention comprises the active EIA gene. The recombinant adenovirus carrying the active EIA gene is replication competent. According to a more preferred embodiment, the recombinant adenovirus comprises the inactive EIB 19 gene and active EIA gene. Still more preferably, the recombinant adenovirus of this invention comprises the inactive EIB 19 gene and active EIA gene, and the chimeric decoy receptor gene in a deleted E3 region.

According to the most preferred embodiment, the recombinant adenovirus of this invention comprises the inactive EIB gene and mutated active EIA gene, and the chimeric decoy receptor gene in a deleted E3 region. The mutated active EIA gene refers to EIA region having a mutated Rb (retinoblastoma protein) binding region in which a Glu residue positioned at amino acid 45 of the Rb-binding region is substituted with a Gly residue and all of amino acids positioned at amino acids 121-127 of the Rb-binding region are substituted with Gly residues.

It has been already suggested that tumor cells have mutated Rb and impaired Rb-related signal pathway as well as mutated p53 protein. Hence, the replication of adenoviruses lacking Rb binding capacity is suppressed in normal cells by virtue of Rb activity, whereas adenoviruses lacking Rb binding capacity actively replicate in tumor cells with repressed Rb activity to selectively kill tumor cells. In this context, the recombinant adenoviruses with the mutated Rb binding region show significant tumor specific oncolytic activity.

As demonstrated in Examples described hereunder, the recombinant adenovirus of this invention expressing the chimeric decoy receptor selectively inhibits angiogenesis by VEGF, particularly angiogenesis of tumor cells by VEGF, thereby exhibiting dramatic antitumoric effects. In addition, the recombinant adenovirus of this invention expressing the chimeric decoy receptor exhibits higher tumoricidal effects even in a lower dose, resulting in excellent safety in body.

In another aspect of this invention, there is provided an anti-angiogenesis composition comprising: (a) a therapeutically effective amount of the recombinant adenovirus of the present invention described above; and (b) a pharmaceutically acceptable carrier.

In still another aspect of this invention, there is provided a method for preventing or treating a disease caused by excessive angiogenesis, comprising administering an anti-angiogenesis composition comprising: (a) a therapeutically effective amount of the recombinant adenovirus of the present invention described above; and (b) a pharmaceutically acceptable carrier to a subject in need thereof.

Since the recombinant adenovirus contained as active ingredients in the pharmaceutical composition is identical to the recombinant adenovirus of this invention described above, the detailed descriptions of the recombinant adenovirus indicated above are common to the pharmaceutical composition. Therefore, the common descriptions between them are omitted in order to avoid undue redundancy leading to the complexity of this specification.

The diseases or disorders prevented or treated by the anti-angiogenesis composition includes any diseases or disorders caused by excessive angiogenesis, preferably, cancer, tumor, diabetic retinopathy, retinopathy of prematurity, corneal transplant rejection, neovascular glaucoma, erythrosis, proliferative retinopathy, psoriasis, hemophilic arthropathy, proliferation of capillaries in atherosclerotic plaques, keloid, wound granulation, vascular adhesion, rheumatoid arthritis, osteoarthritis, autoimmune disease, Crohn's disease, recurrent stricture, atherosclerosis, intestinal tract adhesion, cat scratch disease, ulcer, hepatocirrhosis, glomerulonephritis, diabetic nephropathy, malignant nephrosclerosis, thrombotic microangiopathy, organ transplant rejection, glomerulopathy, diabetes, inflammation and neurodegerative disease.

The recombinant adenovirus expressing the chimeric decoy receptor exhibits dramatic therapeutic effects on various angiogenesis-related diseases, particularly cancers, by effectively inhibiting angiogenesis. In addition, where the recombinant adenovirus has the inactive E1B 55 gene or the mutated Rb binding sites in the E1A, its specificity to cancer cells is significantly high. For these reasons, the titer of viruses for cancer treatment becomes reduced and in vivo toxicity and immune reactions by viruses becomes much lower.

Since the recombinant adenovirus contained the pharmaceutical composition has oncolytic effect to a wide variety of tumor cells, the pharmaceutical composition of this invention is useful in treating tumor-related diseases, including stomach cancer, lung cancer, breast cancer, ovarian cancer, liver cancer, bronchogenic cancer, nasopharyngeal cancer, laryngeal cancer, pancreatic cancer, bladder cancer, colon cancer, and uterine cervical cancer. The term “treatment” as used herein, refers to (i) suppression of disease or disorder development; (ii) alleviation of disease or disorder; and (iii) curing of disease or disorder. Therefore, the term “therapeutically effective amount” as used herein means an amount sufficient to achieve the pharmaceutical effect described above.

The pharmaceutically acceptable carrier contained in the pharmaceutical composition of the present invention, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative.

The pharmaceutical composition according to the present invention may be preferably administered parenterally, i.e., by intravenous, intraperitoneal, intratumoral, intramuscular, subcutaneous, intracardiomuscular or local administration. For example, the pharmaceutical composition may be administered intraperitoneally to treat ovarian cancer and intravenously to treat liver cancer, directly injected to visible tumor mass to treat breast cancer, directly injected to enema to treat colon cancer, and directly injected to a catheter to treat bladder cancer.

A suitable dosage amount of the pharmaceutical composition of the present invention may vary depending on pharmaceutical formulation methods, administration methods, the patient's age, body weight, sex, pathogenic state, diet, administration time, administration route, an excretion rate and sensitivity for a used pharmaceutical composition, and physicians of ordinary skill in the art can determine an effective amount of the pharmaceutical composition for desired treatment.

Generally, the pharmaceutical composition of the present invention comprises 1×10⁵-1×10¹⁵ pfu/ml of a recombinant adenovirus, and 1×10¹⁰ pfu of a recombinant adenovirus is typically injected once every other day over two weeks.

According to the conventional techniques known to those skilled in the art, the pharmaceutical composition comprising the recombinant adenovirus according to the present invention may be formulated with pharmaceutically acceptable carrier and/or vehicle as described above, finally providing several forms a unit dose form and a multi-dose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

The pharmaceutical composition comprising the recombinant adenovirus according to the present invention may be utilized alone or in combination with typical chemotherapy or radiotherapy. Such combination therapy may be more effective in treating cancer. The chemotherapeutic agents useful for the combination therapy include cisplatin, carboplatin, procarbazine, mechlorethamine, cyclophosphamide, ifosfamide, melphalan, chlorambucil, bisulfan, nikosourea, dactinomycin, daunorubicin, doxorubicin, bleomycin, plicomycin, mitomycin, etoposide, tamoxifen, taxol, transplatinum, 5-fluorouracil, vincristin, vinblastin and methotrexate. Examples of the radiotherapy useful for the combination therapy include X-ray illumination and γ-ray illumination.

### Advantageous Effects

The features and advantages of the present disclosure may be summarized as follows:

(a) The recombinant adenovirus of the present disclosure expresses a chimeric decoy receptor which inhibits angiogenesis.

(b) The recombinant adenovirus of the present disclosure which expresses the chimeric decoy receptor may be used for gene therapy of various angiogenesis-related diseases since it inhibits angiogenesis very effectively.

(c) In particular, the recombinant adenovirus of the present disclosure has superior oncolytic activity.

(d) Whereas the existing angiogenesis-related anticancer agents (e.g., Avastin) are limited for cancer treatment since they have only cytostatic effects, the recombinant adenovirus of the present disclosure is capable of killing cancer cells and thus can overcome the limitation of the existing anticancer agents.

(e) And, whereas the existing angiogenesis-related anticancer agents induce side effects by acting on normal cells, the recombinant adenovirus of the present disclosure acts specifically on cancer cells.

(f) Whereas the existing protein-based VEGF trap is short-lived in living organisms, the recombinant adenovirus of the present disclosure can solve this problem since it continuously overexpresses the VEGF trap.

### DESCRIPTION OF DRAWINGS

FIGS. 1a-1b show constructs of recombinant adenoviral (Ad) vectors. FIG. 1a shows an E1-deficient, replication-incompetent adenovirus. dE1-k35 expresses β-galactosidase under regulation by the cytomegalovirus (CMV) promoter. dE1-k35/KH903 comprises a chimeric decoy receptor KH903 in the E3 region. FIG. 1b shows a replication-competent adenovirus. RdB comprises mutated E1A and has E1B 19 and 55 kDa deleted. RdB/KH903 comprises a chimeric decoy receptor KH903 in the E3 region.

FIG. 1c shows a result of detecting KH903 secreted to culture medium (Ad: adenovirus; ITR: inverted terminal repeat).

FIGS. 2a-2b show a result of quantifying VEGF level which is indicative of inhibition of VEGF expression by dE1-k35/KH903. In FIG. 2a, various human lung cancer cell lines were infected with 20-100 MOI dE1-k35 or dE1-k35/KH903. 48 hours after the infection, the concentration of VEGF in the supernatant was determined by ELISA. FIG. 2b shows a result of measuring VEGF level in A549 cell lysate.

FIG. 3 shows a result of testing inhibition of VEGF-induced proliferation of HUVECs by dE1-k35/KH903. HUVECs were treated with 30 MOI dE1-k35 or dE1-k35/KH903. 72 hours after the infection, Cell viability was measured by MTT assay. Average of three repeated experiments is shown.

FIGS. 4a-4b show the effect of dE1-k35/KH903 on migration of HUVECs. The cells were placed into an upper chamber of a 24-well tissue culture plate containing EBM. 3.5 hours later, passed cells were fixed and stained with hematoxylin and eosin (H&E). FIG. 4a shows migration of HUVECs (×40). In FIG. 4b, migrated cells were counted per high power field (×200). 8 fields were counted twice per each. The error bars show ±standard error (*: P<0.05, **: P<0.001).

FIGS. 5a-5b show the effect of dE1-k35/KH903 on tube formation of HUVECs. HUVECs were plated on a Matrigel-coated plate at 1.5×10⁵ cells/well and then cultured for 48 hours using dE1-k35- or dE1-k35/KH903-infected (20 MOO A549 or H460 conditioning medium. FIG. 5a shows representative images of tube formation (×40). FIG. 5b shows a quantitative analysis of tube formation. The tube formation was quantified by measuring the area covered by tube network. Experiment was performed 3 times and average was shown. The error bars show±standard error (*: P<0.05, **: P<0.001).

FIG. 6 shows inhibition of blood vessel sprouting by dE1-k35/KH903. The replication-incompetent adenovirus carrying KH903 inhibits VEGF-induced blood vessel sprouting ex vivo. The analysis result was scored from 0 (minimum positive) to 5 (maximum positive).

FIG. 7 shows the cytopathic effect of RdB/KH903 in vitro. Cells were infected with dE1-k35, dE1-k35/KH903, RdB or RdB/KH903 of predetermined MOI. The replication-incompetent adenovirus dE1-k35 was used as negative control. On days 4-10 after the infection, the cells in the plate were fixed and stained with crystal violet.

FIG. 8 shows the antitumor effect of KH903-expressing adenovirus. A xenograft model was established by subcutaneously injecting 1×10⁷ H460 tumor cells. The tumor was allowed to grow to 80-120 mm³. Nude mice bearing the tumor were randomly divided into 3 groups (5 mice per each). For each test group, adenovirus (1×10¹⁰ vp of adenovirus in 30 μL of PBS) was injected into the tumor on days 1, 3 and 5. Tumor growth was monitored every day by measuring minor axis (w) and major axis (L).

FIGS. 9a-9b show a histological evaluation result of H460 tumor tissue treated with RdB/KH903. FIG. 9a shows microvessels stained with anti-PECAM antibody (CD31). Tissues stained with CD31 are shown. FIG. 9b shows a result of quantifying the number of blood vessels in tumor tissue. Data are given as mean (n=3)±standard error.

### MODE FOR INVENTION

The examples and experiments will now be described. The following examples and experiments are for illustrative purposes only and not intended to limit the scope of the present disclosure.

### EXAMPLES

Test Materials and Methods

1. Cell Lines and Cell Culture

Human lung cancer cell lines A549 and H460 were acquired from the American Type Culture Collection (ATCC; Manassas, Va., USA) and human umbilical vascular endothelial cells (HUVECs) were purchased from Lonza (Basel, Switzerland). HEK293 cells (ATCC) with the adenovirus early gene E1 inserted in the host genome were used to produce adenovirus. All other cell lines excluding HUVECs were cultured in DMEM containing 10% fetal bovine serum (FBS; Gibco-BRL, Grand Island, N.Y., USA) as well as 100 U/mL penicillin and 100 μg/mL streptomycin (Gibco-BRL) as antibiotics in a 37° C. incubator in the presence of 5% CO₂. HUVECs were cultured in EGM-2MV (Lonza, Walkersville, Md., USA) containing 5% FBS as well as 100 U/mL penicillin and 100 μg/mL streptomycin (Gibco-BRL) as antibiotics. Cells of 5-8 passages were used.

2. Production and Titration of KH903-Expressing Adenoviruses

In order to construct a KH903-expressing recombinant adenovirus, pKH903 (KangHong, Chengdu, China) which is a KH903 plasmid was inserted into the adenovirus E1 shuttle vector pCA14 (Microbix) by EcoRI digestion, which was then digested with Bg/II. The resulting KH903 DNA fragment was inserted into the E3 shuttle vector pSP72ΔE3 created by the inventors of the present disclosure (Cancer Gene Therapy, 12: 61-71 (2005)) by BamHI digestion. KH903 was constructed by fusing the human IgG Fc region (SEQ ID NOS 7 and 8) with a chimeric decoy receptor prepared by sequentially attaching the second extracellular domain of VEGFR-1 (SEQ ID NOS 1 and 2), the third extracellular domain of VEGFR-2 (SEQ ID NOS 3 and 4) and the fourth extracellular domain of VEGFR-2 (SEQ ID NOS 5 and 6). The constructed pSP72ΔE3/KH903 vector was digested by XbaI and the CMV promoter of the pSP72ΔE3/CMV vector created by the inventors of the present disclosure (Cancer Gene Therapy, 12: 61-71 (2005)) was inserted to prepare the pSP72ΔE3-CMV-KH903 E3 shuttle vector. In order to construct a KH903-expressing replication-incompetent adenovirus, the pSP72ΔE3-CMV-KH903 E3 shuttle vector was linearized by treating with PvuI. And, the pdE1-k35 total vector with the E3 gene deleted, lacZ inserted in the E1 region and substituted with the adenovirus type 35 fiber knob (knob) [700-bp 35 knob was obtained from adenovirus having Ad35 fiber knob (Cell Genesys) by PCR, digested with NcoI/MfeI and ligated with pSK5543 (Coxsackie and adenovirus receptor binding ablation reduces adenovirus liver tropism and toxicity, Human Gene Ther 16: 248-261 (2005)) which had been digested with NcoI/MfeI to construct pSK5543/35k. Thus obtained pSK5543/35k was digested with SacII/XmnI and homologously recombined with dE1/lacZ that had been digested with SpeI to construct pdE1-k35.] was linearized by treating with the restriction enzyme SpeI. They were cotransformed into E. coli BJ5183 (obtained from Dr. Verca, University of Fribourgh, Switzerland; Heider, H. et al., Biotechniques, 28(2): 260-265, 268-270 (2000)) to induce homologous recombination, finally constructing pdE1-k35/KH903 which is a replication-incompetent adenoviral vector expressing both the lacZ gene and KH903. To construct an oncolytic adenovirus expressing the VEGF trap capable of effectively inhibiting VEGF, the pSP72ΔE3-CMV-KH903 E3 shuttle vector was linearized by treating with PvuI and then cotransformed into E. coli BJ5183 together with the SpeI-digested pRdB adenovirus total vector (oncolytic adenovirus having mutated Rb binding site in E1A and 19-kDa E1B gene and 55-kDa E1B gene deleted; see Korean Patent No. 0746122), generating the pRdB/KH903 oncolytic adenoviral vector. The mutation of the Rb binding site in E1A is substitution of the 45th Glu residue of the nucleotide sequence coding for the Rb binding site of the E1A gene with Gly and substitution of the 121st through 127th amino acids with Gly. The homologously recombined adenoviral vectors were digested with the restriction enzyme HindIII to confirm the homologous recombination and the confirmed plasmids were digested with PacI, followed by transforming into HEK293 cells to produce adenoviruses. As control viruses, dE1-k35 having the lacZ gene in deleted E1 region and RdB having both the 19-kDa E1B gene and 55-kDa E1B gene deleted were used. Each adenovirus was proliferated in HEK293 cells, concentrated using CsCl gradient and then purified. Titers (plaque forming unit; PFU) were analyzed by limiting titration assay using a photospectrometer.

3. Western Blotting

In order to verify whether KH903 protein is produced and secreted from human lung cancer cells infected with the KH903-expressing adenovirus, A549 cells were treated with 20, 50 or 100 MOI of the dE1-k35/KH903 adenovirus. 48 hours later, the cells were collected and subjected to sodium dodecyl sulfate polyacrylamide gel electrophoresis (SDS-PAGE). After the electrophoresis, the proteins remaining on the gel were electrotransferred onto polyvinylidene fluoride (PVDF) membrane and probed with an antibody specifically recognizing the human IgG Fc region of KH903 as primary antibody (Cell signaling, Danvers, Mass., USA). After reacting with horseradish peroxidase (HRP)-conjugated goat anti-mouse IgG as secondary antibody (Cell signaling, Danvers, Mass., USA), protein-antibody binding was examined by enhanced chemiluminescence (ECL) (Pierce, Rockford, Ill., USA) using LAS4000 and expression level of each protein was determined.

4. Change in VEGF Expression

Enzyme-linked immunosorbent assay (ELISA) was conducted in order to verify whether the expression of VEGF from tumors can be effectively inhibited by the KH903-expressing adenovirus. First, to verify the suppression of VEGF expression, lung cancer cell lines A549, H460, H322 (ATCC), H358 (ATCC) and H1299 (ATCC) were transferred to a 6-well plate with 3×10⁵ cells/well. The next day, they were infected with adenovirus at 2-100 multiplicity of infection (MOI) and medium was changed with fresh DMEM containing 5% FBS 6 hours later. In order to collect the medium 48 hours after the viral infection, the medium was changed with FBS-free DMEM 24 hours prior to the collection. The collected medium was centrifuged at 800×g. The supernatant was separated and 150 μg was subjected to VEGF ELISA analysis.

5. MTT Assay

MTT (3-(4,5-dimethylathiazol-2yl)-2,5-diphenyltetrazolium bromide, 2 mg/mL) assay was conducted to quantify the suppressed proliferation of vascular endothelial cells by KH903 expression due to the adenoviral infection. HUVECs were plated on a 2% gelatin-coated 48-well plate and treated with 30 MOI of the prepared recombinant adenovirus 24 hours later. Prior to treatment with the virus, HUVECs were serum starvation-pretreated using EBM-2 (Lonza, Walkersville, Md., USA). 72 hours after the viral treatment, the medium was removed and 150 μL of MTT solution was added to each well to determine cell viability. After incubating in a 5% CO₂ incubator at 37° C. for 4 hours, the supernatant was removed. Then, after adding 1 mL of dimethyl sulfoxide (DMSO) to each well, followed by incubation at 37° C. for 10 minutes, absorbance of the supernatant was measured at 540 nm to determine relative viability.

6. Migration of Endothelial Cells

Endothelial cell migration assay was performed using Transwell (Corning Costar, Cambridge, Mass., USA) with 6.5-mm diameter polycarbonate filter paper (8-μm pore size) in order to determine the chemotactic motility of HUVECs. First, 0.1% gelatin was coated on the filter of the upper chamber. After the gelatin is completely dried, HUVECs were serum-starved by preincubating in serum-free medium for 6 hours. 1×10⁵ HUVECs were placed in the upper chamber and infected with the dE1-k35 and dE1-k35/KH903 adenoviruses. The collected cell culture was placed in the lower chamber and the plate was incubated at 37° C. for 3 hours and 30 minutes. Then, after removing medium from the upper chamber, the cells were fixed for 1 minute with methanol and stained with H&E. Thus prepared slides were photographed at ×200 magnification for 8 regions. The cell motility was quantified by averaging.

7. Tube Formation Assay

To verify whether the tube formation of vascular endothelial cells is altered by decreased VEGF expression by KH903 which is capable of effectively inhibiting VEGF secreted from tumors, tube formation assay was performed using HUVECs. First, 250 μL of growth factor-reduced Matrigel (Collaborative Biomedical Products, Bedford, Mass., USA) was uniformly plated onto a 24-well plate, which had been kept at −20° C., for 30 minutes at 37° C. HUVECs (5-7 passage cultures) were cultured for 6 hours in serum-free EBM-2 (Lonza, Walkersville, Md., USA) to until serum starvation. After treating with trypsin, the cells were counted. After treating with 20 MOI dE1-k35 or dE1-k35/KH903 adenovirus for 48 hours, the resulting A549 and H460 cell cultures were mixed with serum starvation-pretreated HUVECs (1.5×10⁵ cells/well) and cultured after being plated onto a 24-well plate containing Matrigel. As positive control, 20 ng/mL VEGF protein was used. The cells were removed from the medium between 12 and 16 hours after culturing, washed twice with PBS, and then observed under a microscope.

8. Ex Vivo Aorta Ring Sprouting Assay

To evaluate the suppression of blood vessel formation by KH903 which is capable of effectively inhibiting VEGF secreted from tumors, aorta ring sprouting assay was carried out. The aorta was separated from a 6-week-old Sprague Dawley rat (Orient Bio, Korea, Inc.). After removing the fibro-adipose tissues around the aorta, the aorta was sectioned to 1-mm thick rings. 200 μL of Matrigel was plated on each well of a 48-well plate that had been cooled and the aorta ring was placed on each well. Matrigel was solidified at 37° C. for 20 minutes. 30 minutes later, 250 μL of the cell culture used in the tube formation assay was introduced to each well and incubated. Blood vessels generated from the aorta ring were observed every day under a microscope. As positive control, VEGF protein (20 ng/mL) was used. The newly formed blood vessels were analyzed in a double-blinded manner in which the positive control was scored 5 and no vessel formation was scored 0. The aorta ring sprouting assay was performed with 12 aorta rings for each test group.

9. Cytopathic Effect of KH903-Expressing Tumor-Specific Adenovirus

To assess whether the expression of KH903 which decreases secretion of VEGF from tumors affects the replication of adenovirus, the cytopathic effect (CPE) was analyzed. Human tumor cells including lung cancer cells were plated on a 48-well plate and then infected with 0.1-10 MOI dE1-k35, dE1-k35/KH903, RdB or RdB/KH903 adenovirus 24 hours later. At the moment when the cells infected with the virus exhibited the most prominent difference from the control virus, the medium was removed and the cells remaining on the plate were stained with 0.5% crystal violet and then analyzed.

10. In Vivo Antitumor Effect

1×10⁷ human lung cancer cells H460 were subcutaneously injected into the abdomen of 6-8-week-old nude mice (Orient). When the tumor volume reached about 70-100 mm³, RdB and RdB/KH903 adenoviruses or PBS as negative control were injected directly into the tumors 3 times every other day. The tumor volume was measured every other day using a caliper. The tumor volume was calculated using the following formula: tumor volume (mm³)=(minor axis (mm))²×major axis (mm)×0.523.

11. Suppression of Angiogenesis in Tumor Tissue by KH903-Expressing Tumor-Specific Oncolytic Adenovirus That Binds With VEGF

Lung cancer cells H460 were subcutaneously injected into the abdomen of 6-8-week-old nude mice. When the tumor size reached about 100-120 mm³, RdB and RdB/KH903 adenovirus or PBS as negative control were injected into the tumors 3 times every other day. About 10 days after the final injection of virus, tumors were isolated and fixed in IHC zinc fixative (formalin-free) (BD Biosciences Pharmingen, San Diego, Calif., USA) in order to prepare paraffin blocks. The prepared paraffin blocks were cut into 4-μm thick slices and immersed successively in xylene and 100%, 95%, 80% and 70% ethanol for deparaffinization, followed by staining with were hematoxylin and eosin (H&E). In order to elucidate whether angiogenesis in tumor tissues were suppressed by KH903 that decreases expression of VEGF secreted from tumors by binding therewith, immunohistochemical staining was performed using rat anti-mouse CD31 monoclonal antibody (MEC13.3; BD Biosciences Pharmingen) which is capable of specifically recognizing the vascular endothelial cell-specific antigen CD31. The paraffin-removed 4-μm thick tumor tissue slide was incubated in 3% H₂O₂ solution for 10 minutes to block the action of endogenous peroxidase. Then, after blocking non-specific antibody reactions by incubating with Protein Block Serum-Free (DakoCytomation, Carpinteria, Calif., USA) for 30 minutes, hybridization was performed with CD31 antibody as primary antibody. Then, the slide was incubated with biotin-conjugated polyclonal anti-rat IgG antibody (BD Biosciences Pharmingen) as secondary antibody and the expression of CD31 was determined using DAB (DakoCytomation, Carpinteria, Calif., USA).

12. Counting of Blood Vessels in Tumor

Blood vessels stained with the vascular endothelial cell-specific antigen CD31 (platelet endothelial cell adhesion molecule 1) were observed at low magnification and photographs were obtained randomly. Then, the number of the blood vessels was determined at ×100 magnification. From three slides, 5 visual fields were selected and the number of blood vessels was determined. The mean value was calculated as representative value.

Results

1. Production of KH903-Expressing Adenoviruses Binding Specifically to VEGF and Evaluation of VEGF Expression

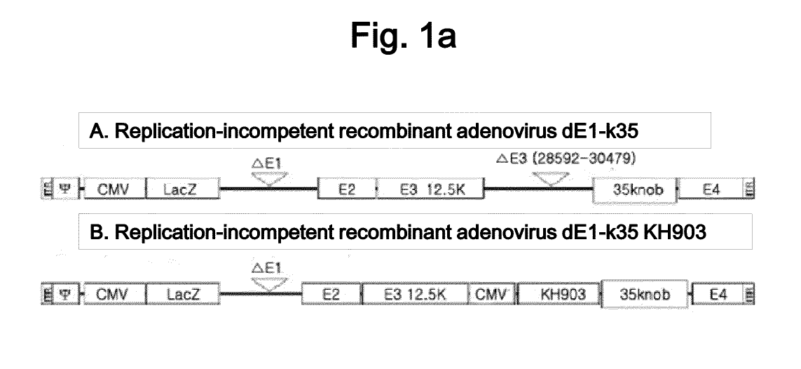

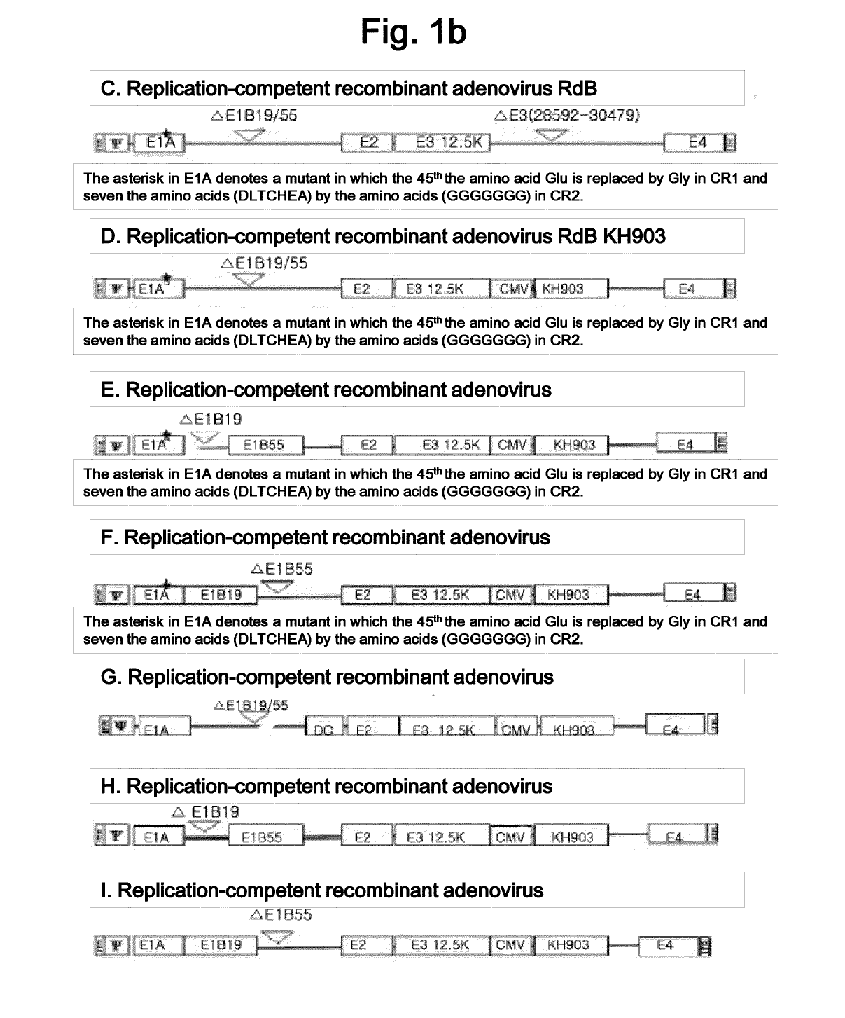

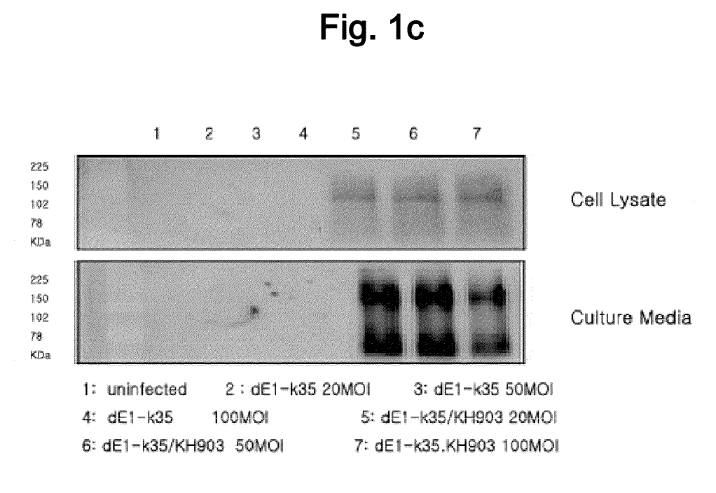

The KH903-expressing adenovirus dE1-k35/KH903 which is a VEGF trap that binds specifically to VEGF and thus inhibits expression of VEGF secreted from tumors was constructed (FIG. 1a). In order to identify whether the KH903 inserted into the E3 region of the dE1-k35/KH903 adenovirus is actually secreted from the infected cells, western blotting was conducted for cell lysate and culture medium using an antibody that can detect the Fc region of human IgG of KH903. As a result, a large amount of KH903 was observed in the culture medium whereas KH903 was only detectable in the cell lysate. Thus, it was identified that KH903 is produced in the infected cells and secreted to the culture medium (FIG. 1c).

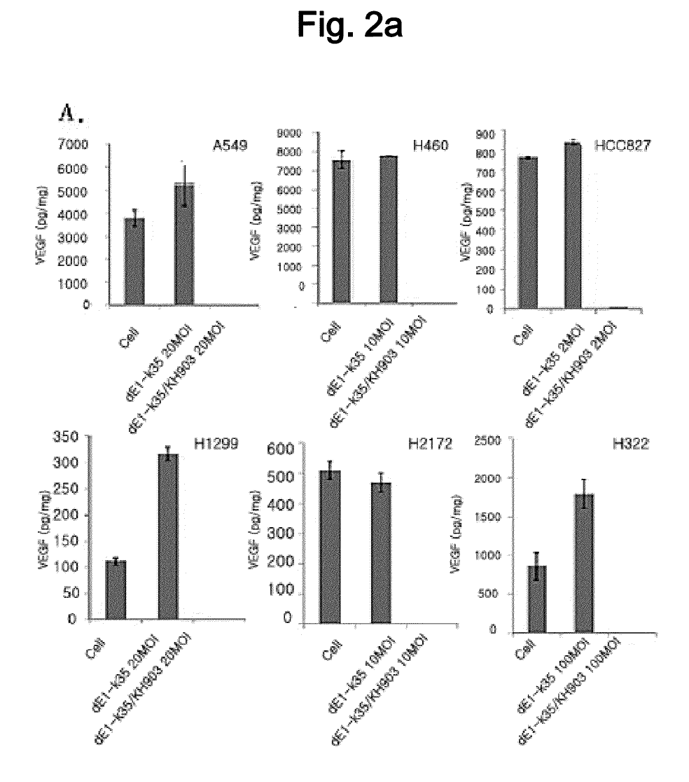

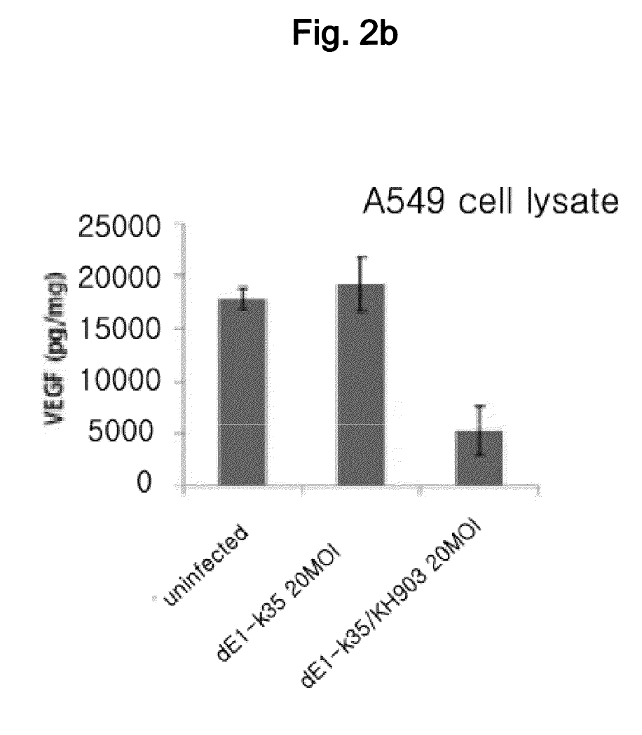

Because it was reported that replication-competent adenoviruses expressing the adenoviral early gene E1A could suppress VEGF²⁸, dE1-k35/KH903 which is a replication-incompetent adenovirus lacking E1A and expressing both lacZ and KH903 was constructed to verify the change in VEGF expression by KH903. Human lung cancer cells (A549, H460, HCC827, H1299, H2172 and H322) were infected with dE1-k35/KH903 and culture medium was collected for ELISA analysis to quantify VEGF expression. It was revealed that VEGF expression was significantly decreased in all the lung cancer cancers by the dE1-k35/KH903 adenovirus (FIG. 2a).

In order to investigate how much VEGF is produced actually in the tumor cells and how much it is decreased by the KH903 expression, the VEGF expression level was determined for the cell lysate. As seen from FIG. 2b, the VEGF expression level was significantly decreased in the cells infected with dE1-k35/KH903 as compared to those infected with dE1-k35.

2. Suppression of Angiogenesis by KH903-Expressing Adenovirus Binding Specifically to VEGF

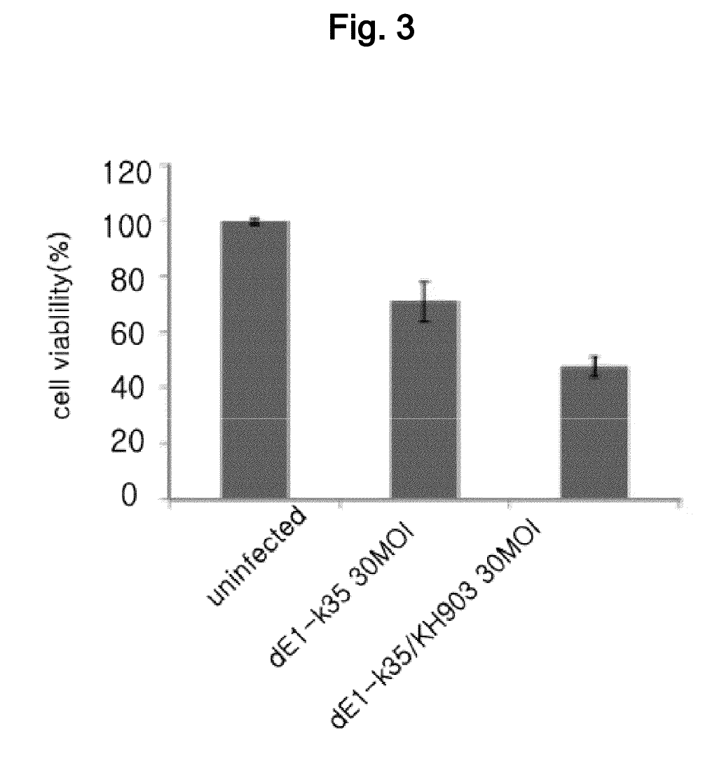

First, the influence of change in VEGF level by the expression of KH903 inhibiting VEGF on VEGF-induced proliferation of HUVECs was investigated. HUVECs were seeded on a Matrigel-coated 48-well plate at 2×10⁴ cells/well and then infected with 30 MOI of the dE1-k35 or dE1-k35/KH903 adenovirus. 72 hours later, cell viability was measured by MTT assay. As a result, the group infected with dE1-k35/KH903 showed 53% decreased viability as compared to non-treated group. The group treated with the positive control dE1-k35 showed a decrease of 30% (FIG. 3).

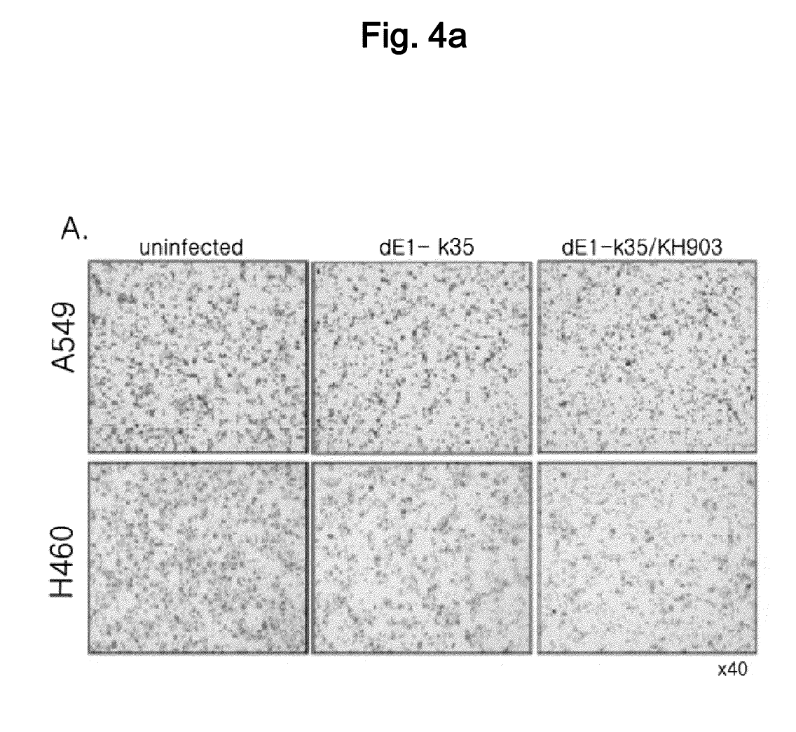

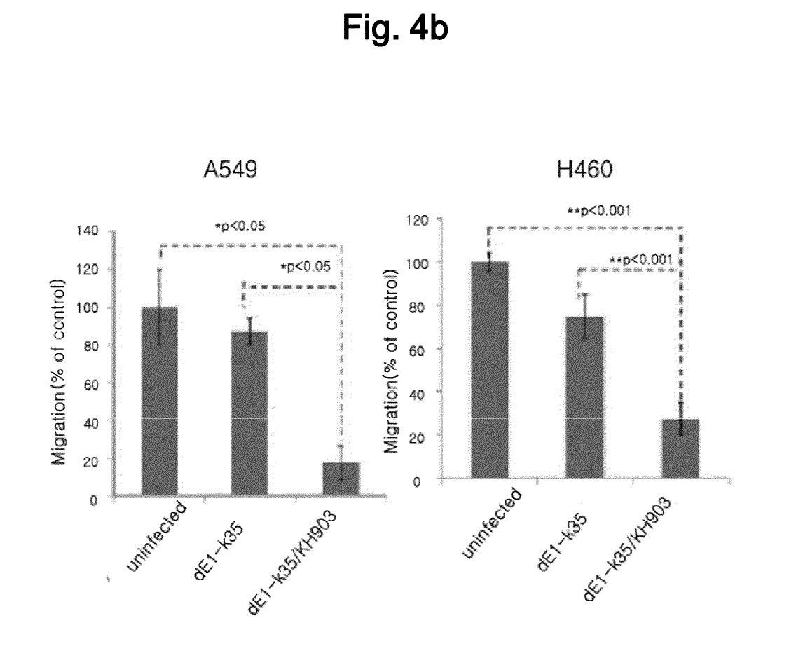

In order to verify the influence of change in VEGF level by the KH903 inhibiting VEGF expression on the motility of vascular endothelial cells, migration assay was conducted using HUVECs. A549 and H460 cells were infected with 20 MOI of the dE1-k35 or dE1-k35/KH903 adenovirus. Then, HUVECs were cultured using the medium obtained 48 hours later. As a result, whereas a lot of the HUVECs migrated from the upper chamber to the lower chamber when they were non-treated or infected with the dE1-k35 adenovirus, those infected with the dE1-k35/KH903 adenovirus showed less migration as compared to the two groups (FIG. 4).

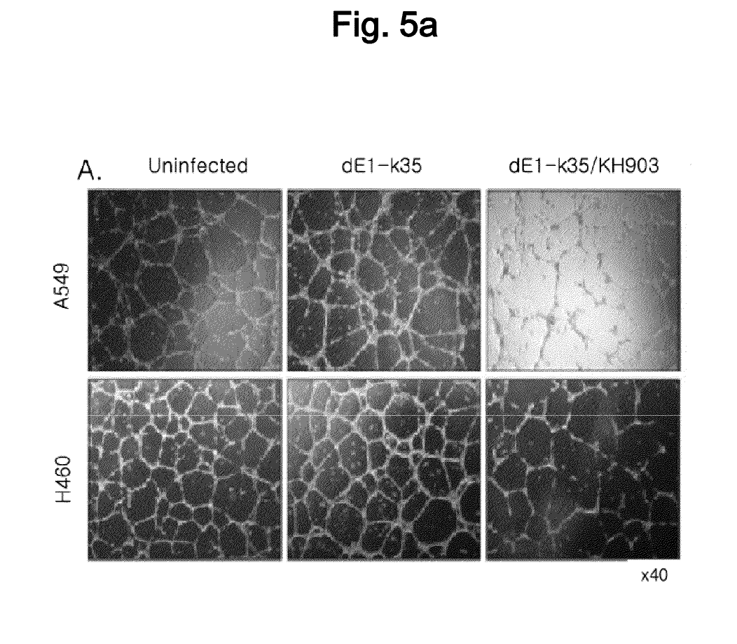

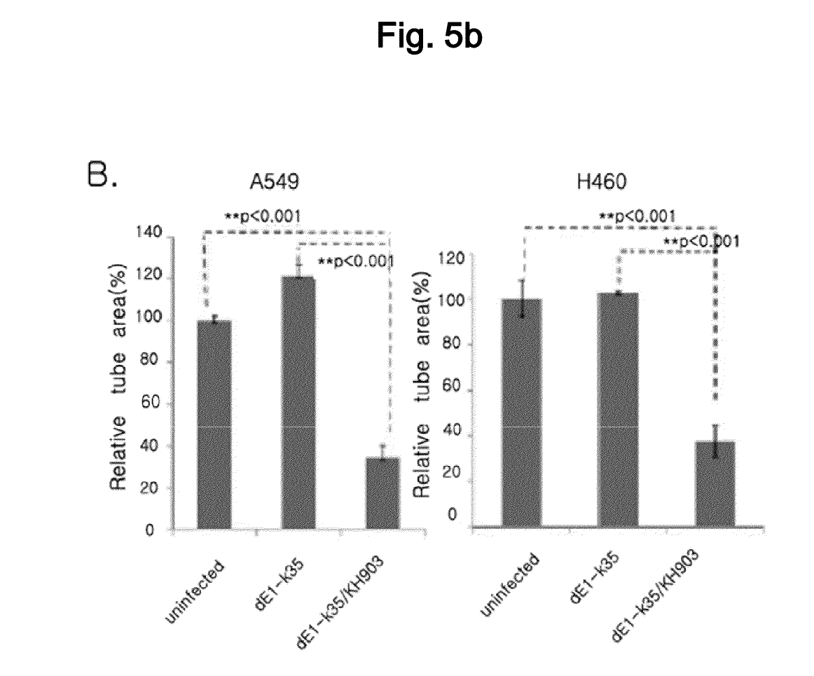

In order to verify the influence of change in VEGF level by KH903 expression on the blood vessel forming ability of vascular endothelial cells, tube formation assay was performed using HUVECs. A549 and H460 cells were infected with 20 MOI of the dE1-k35 or dE1-k35/KH903 adenovirus. Then, HUVECs were cultured using the medium obtained 48 hours later. As a result, whereas the HUVECs non-treated or infected with the dE1-k35 adenovirus generated large and thick tubes, those infected with the dE1-k35/KH903 adenovirus formed thinner and partially broken tubes (FIG. 5).

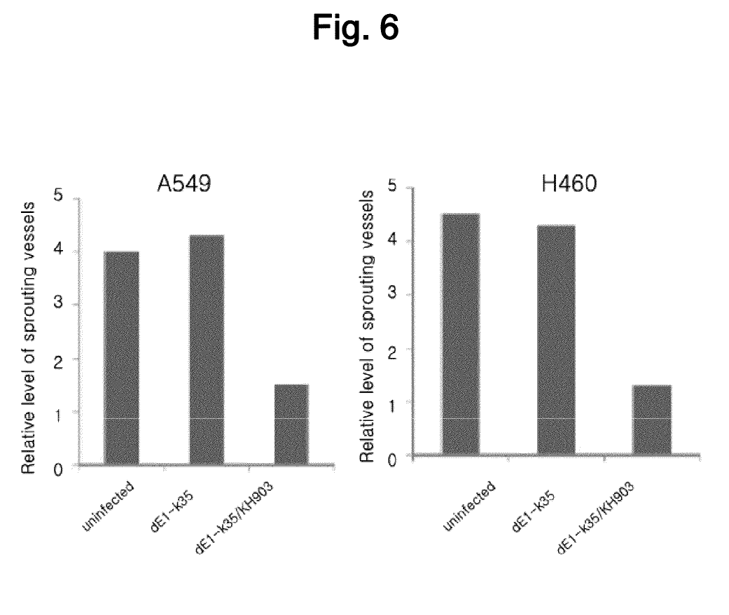

In order to confirm the difference in angiogenesis potentials evaluated above ex vivo, blood vessel sprouting was performed using the rat aorta: First, A549 and H460 cells were infected with 20 MOI of the dE1-k35 or dE1-k35/KH903 adenovirus. Then, the aorta ring was incubated with the cell culture obtained 48 hours later for 5 days. As a result, it was observed that the aorta ring incubated in the cell culture treated with the dE1-k35/KH903 adenovirus exhibited little blood vessel sprouting, unlike the aorta ring non-treated or infected with the dE1-k35 adenovirus (FIG. 6). In order to quantitatively confirm the vessel sprouting potentials, the vessels formed were analyzed in a double-blinded manner in which the positive control group (most positive) was scored 5 and the non-sprouting test group (least positive) was scored 0. It was confirmed more active vessel sprouting occurred in the aorta ring non-treated or treated with the A549 or H460 cell culture infected with dE1-k35 as compared to that treated with the cell culture infected with the dE1-k35/KH903 adenovirus, indicating that the tube formation is remarkably suppressed as compared to the control virus dE1-k35.

3. Cytopathic Effect of KH903-Expressing Oncolytic Adenovirus Binding Specifically to VEGF

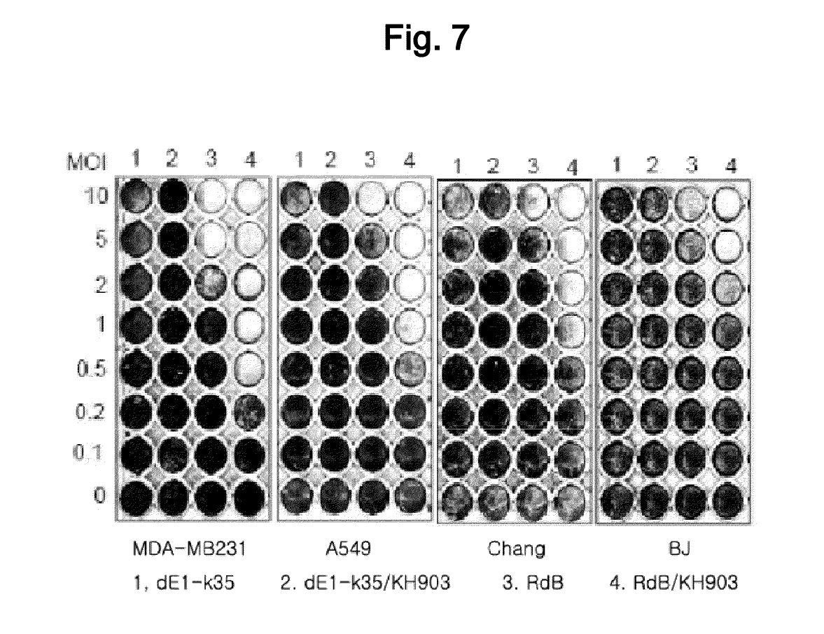

Since the decrease in angiogenesis potential by suppression of VEGF expression can lead to suppressed tumor growth, the oncolytic adenovirus RdB/KH903 expressing KH903 and the oncolytic adenovirus RdB as control were constructed to investigate the anticancer effect of KH903. First, in order to verify whether the expression of KH903 inhibits replication of adenoviruses, various cancer cells and normal cells were infected with dE1-k35, dE1-k35/KH903, RdB or RdB/KH903 and CPE assay was performed to analyze cell lysis due to viral replication. Since the adenoviral replication does not occur in cells infected with the negative control dE1-k35 replication-incompetent adenovirus, the cytopathic effect was not detected. However, when the cells were infected with the replication-competent adenovirus RdB or RdB/KH903, the cytopathic effect increased as the titer of the virus increased. In particular, the KH903-expressing adenovirus RdB/KH903 showed excellent cytopathic effect in all the cell lines tested as compared to the control virus RdB (FIG. 7).

4. In Vivo Antitumor Effect of KH903-Expressing Oncolytic Adenovirus Binding Specifically to VEGF

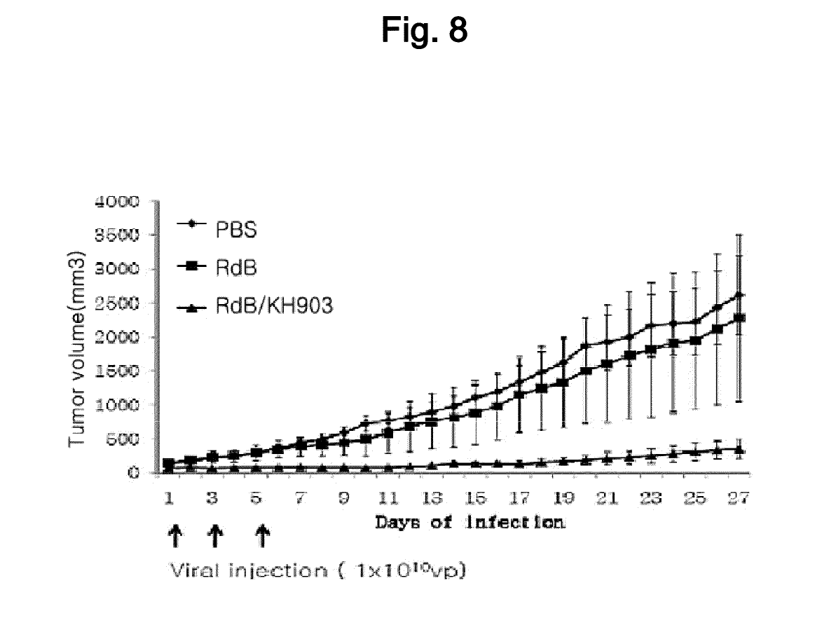

In order to verify the in vivo antitumor effect of the KH903-expressing adenovirus that inhibits VEGF expression, human lung cancer cells H460 were subcutaneously injected into the abdomen of nude mice. When the tumor volume reached about 80-100 mm³, 1×10¹⁰ vp of the RdB or RdB/KH903 adenovirus or PBS as negative control was administered intratumorally 3 times every other day and tumor growth was observed (FIG. 8). Tumor volume increased abruptly to about 2170.238±455.1216 mm³ on day 23 post-treatment in the nude mice treated with the negative control PBS, whereas the tumor growth was substantially delayed when the KH903-expressing oncolytic adenovirus RdB/KH903 was administered. The mice administered with the RdB and RdB/KH903 adenoviruses showed a tumor volume of 1181.391±985.9131 mm³ and 252.67±103.8464 mm³ respectively, evidently showing excellent antitumor effect due to inhibition of angiogenesis by KH903.

5. Change in Blood Vessel Distribution in Tumor by Administration of KH903-Expressing Oncolytic Adenovirus Inhibiting VEGF Expression

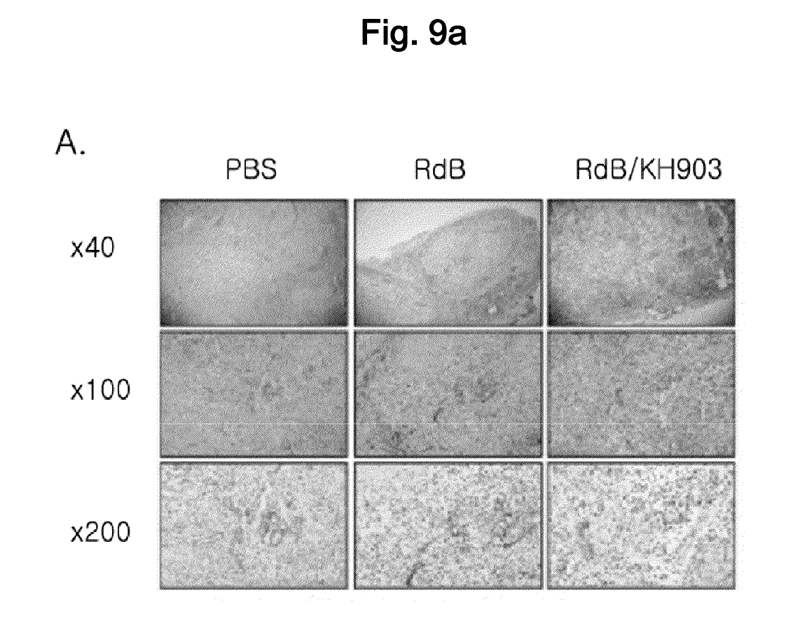

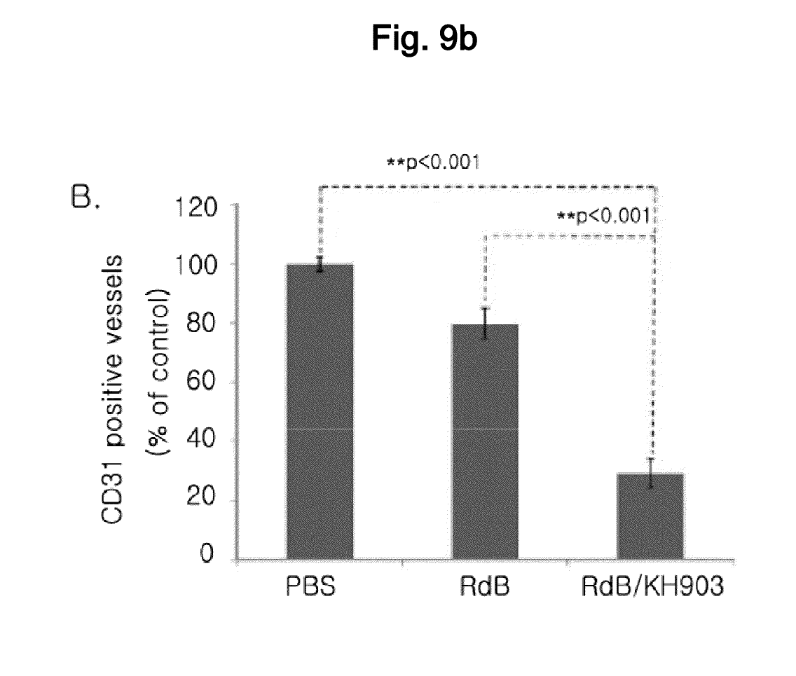

Human lung cancer cells H460 were subcutaneously injected into the abdomen of nude mice. After tumors were formed, 1×10¹⁰ vp of the RdB or RdB/KH903 adenovirus or PBS as negative control was administered intratumorally 3 times every other day. One day after the last administration, the tumors were collected and observed by immunohistochemical staining using the vascular endothelial cell-specific antigen CD31. As a result, the test group treated with the oncolytic adenovirus RdB showed 21% decreased blood vessels in the tumor as compared to the negative control group, whereas the group treated with RdB/KH903 showed 71% decrease (FIG. 9).

Further discussions

Angiogenesis is a process involving the growth of new blood vessels from pre-existing ones and is vital in embryonic development, organ formation and tissue regeneration. Also, angiogenesis is essential in early tumor growth. As the tumor volume increases, the tumor cells or infiltrated macrophages produce various angiogenic factors, thus forming microvessels in tumors. Thus formed blood vessels supply nutrients as well as various growth factors to the tumor cells. Among the various growth factors involved in angiogenesis, vascular endothelial growth factor (VEGF) is known to play typical roles in tumor growth and metastasis. VEGF acts as a potent angiogenic factor by directly binding to two tyrosine receptors VEGFR2 (KDR) and promoting the division of vascular endothelial cells, thereby increasing permeability of microvessels and promoting secretion of serum proteins to nearby tissues and modification of the extracellular matrix. Accordingly, inhibition of the angiogenic factor VEGF is essential to suppress cancer growth. In the last 30 years, suppression of tumor growth by inhibiting angiogenesis in tumors has been actively studied as a target of cancer therapy. However, most of the currently available angiogenesis inhibitors are used in combination therapies rather than alone and are problematic in that they are expensive and may incur toxicity due to repeated administration. In order to overcome these disadvantages, the present disclosure is directed to expressing KH903 which acts as a soluble VEGF-specific decoy receptor in an oncolytic adenovirus, thereby effectively inhibiting VEGF, and improving overall antitumor effect by using the oncolytic adenovirus.

KH903 is a VEGF-specific soluble decoy receptor obtained by fusing the VEGF binding domains of VEGFR1 and VEGFR2 and is capable of effectively inhibiting VEGF secreted from tumor cells. That is to say, the KH903 constructed using the major domains of VEGFR1 and VEGFR2 that are directly involved in the interaction of VEGF and VEGFR is capable of suppressing angiogenesis by binding with the VEGF secreted from tumor cells instead of VEGFR and thus blocking the receptor-ligand interaction^{29,30}.

The early developed VEGF trap is one in which the second domain of VEGFR1 and the third domain of VEGFR2, which are major domains binding with VEGF, are fused with the Fc region of human IgG¹¹. In the present disclosure, KH903 is used, which is capable of binding not only with VEGF-A but also with VEGF-B, VEGF-C and placenta growth factor (PGF) and thus has about 2 times improved VEGF-binding ability as compared to the existing VEGF trap. The reason why KH903 shows superior binding ability for all VEGF families including VEGF-A is because of the addition of the fourth domain of VEGFR2 that maintains strong binding between VEGF and its receptor. Further, since this domain stabilizes the 3-dimensional structure of KH903 and makes it easier to form dimers, KH903 has longer span of life than the existing VEGF trap²⁹. In order to investigate the angiogenesis inhibiting effect of KH903 having such advantages, the replication-incompetent adenovirus dE1-k35/KH903 was constructed by inserting KH903 to the E3 region of an adenovirus having β-galactosidase inserted in the E1 region as reporter gene and lacking the E3 region gene. Various lung cancer cells including A549 and H460 showing active angiogenesis were infected with the adenovirus at various MOIs and VEGF expression was compared. In all the cell lines tested, KH903 exhibited strong effect of inhibiting VEGF expression (FIG. 2). After confirming that KH903 effectively inhibits VEGF expression in tumor cells, it was investigated how the decreased VEGF expression actually influences migration and proliferation of vascular endothelial cells as well as formation and extension of blood vessels in vitro and ex vivo.

First, when the vascular endothelial cells HUVECs were infected with the KH903-expressing replication-incompetent virus dE1-k35/KH903, it was confirmed that the viability of the vascular endothelial cells was decreased due to decreased VEGF expression. Then, migration assay was performed to observe the migration potential of the vascular endothelial cells after infecting them with the KH903-expressing replication-incompetent virus or a control virus or neither. When treated with the control virus having sufficient growth factors or non-treated, the HUVECs showed active migration. In contrast, when the cells were treated with the KH903-expressing virus, the migration of HUVECs decreased significantly due to decreased VEGF expression. It was confirmed through tube formation assay and aorta sprouting assay that tube formation and vessel sprouting are suppressed. Since the inhibition of angiogenesis by KH903 can lead to anticancer effect, the RdB/KH903 adenovirus was constructed by inserting KH903 to the oncolytic adenovirus RdB having the Rb-binding site of the E1A region modified and lacking the E1B region and superior antitumor effect was confirmed in an H460 xenograft model. The oncolytic adenovirus RdB-KH903 induces inhibition of VEGF expression not only be expressing the E1A gene but also through effective and continuous gene transfer, thereby remarkably improving antitumor effect in vivo as compared to the control adenovirus RdB. The effect of RdB/KH903 was confirmed again through the blood vessel distribution in tumor tissues. The tumor tissues treated with the oncolytic adenovirus showed decreased blood vessels as compared to the PBS group, confirming that angiogenesis can be inhibited only with the oncolytic adenovirus. Also, it was confirmed that KH903 can further suppress angiogenesis by effectively inhibiting VEGF.

To conclude, the KH903-expressing oncolytic adenovirus RdB-KH903 constructed in the present disclosure provides significantly improved antitumor effect due to the inhibition of angiogenesis in tumors by the VEGF-specific soluble decoy receptor KH903 and the tumor-specific oncolytic ability of the adenovirus.

KH903 constructed by fusing the VEGF-binding domains of VEGFR1 and VEGFR2 with the Fc region of human IgG can effectively inhibit VEGF secreted from tumor cells. The KH903-expressing oncolytic adenovirus RdB-KH903 provided in the present disclosure is expected to be useful in cancer therapy since it exhibits improved antitumor effect due to the tumor-specific oncolytic ability through tumor-specific oncolytic adenoviral replication as well as the inhibition of VEGF induced by E1A expression and KH903.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

References
1. George D Y, Samuel D, Nicolas W G, John S R, Stanley J, Wiegand et al., Vascular-specific growth factors and blood vessel formation. Nature 2000; 407: 242-8.
2. Gabriele B, Rolf B, Gerald M, Thinneu H V, Takeshi I, Kazuhiko T, et al., Matrix metalloproteinase-9 triggers the angiogenic switch during carcinogenesis. Nat Cell Bio 2000; 2: 737-44.
3. Toren F, and Stephen E E, Gene therapy for vascular disease. FASEB J 1995; 9: 843-51.
4. Janice A N, Eliza V, Dian F, Christian S, Lowrence F B, Michael J D et al., Vascular permeability factor/vascular endothelial growth factor induces lymphangiogenesis as well as angiogenesis. J. Exp. Med. 2002; 196: 1497-1506.
5. Megan E B, Steven A S, Mark G A, Molecular control of lymphangiogenesis. Bioessays 2002; 24: 1030-40.
6. Joyce E O, Dmitry I G, George D S, Ekaterina K, Kelly S P, Sorean N et al., VEGF inhibits T-cell development and may contribute to tumor-induced immune suppression. Blood 2003; 101: 4878-86.
7. Joyce E O, Carbone D P, VEGF as a mediator of tumor-associated immunodeficiency. Immunol. Res. 2001; 23: 263-72.
8. Lee M E, Daniel J H, VEGF-targeted therapy: mechanisms of anti-tumour activity, Nat Rev Cancer 2008; 8: 579-91.
9. Kerbel R S, Tumor Angiogenesis, N Engl J Med 2008; 358: 2039-49.
10. Folkman J, Merler E, Abernathy C, Williams G, Isolation of a Tumor factor responsible for angiogenesis. J. Exp. Med. 1971; 133-275.
11. Shin-Ae L, Seok-Reyol C, Jin-Seok J, Jong-Hun L, Myung-Hwan R, Sang Ock K, Expression of VEGF, EGFR, and IL-6 in Gastric Adenomas and Adenocarcinomas by Endoscopic Submucosal Dissection, Dig Dis Sci 2009; 12.
12. Vosseler S, Mirancea N, Bohlen P, Mueller M M, Fusenig N E, Angiogenesis inhibition by vascular endothelial growth factor receptor-2 blockade reduces stromal matrix metalloproteinase expression, normalizes stromal tissue, and reverts epithelial tumor phenotype in surface heterotransplants. Cancer Res 2005; 65: 1294-305.
13. Jocelyn H, Sam D, Nick P, Susan D C, Lillian H, Michelle R et al., VEGF-Trap: a VEGF blocker with potent antitumor effects, Proc Natl Acad Sci USA 2002; 99: 11393-8.
14. S. Percy Ivy, Jeannette Y. Wick and Bennett M K, An overview of small-molecule inhibitors of VEGFR signaling, Nat. Rev. Clin. Oncol. 2009 6: 569579.
15. Ke Xie, Rui-Zhen B, Yang W, Quan L, Kang L, Yu-Quan W, Anti-tumor effects of a human VEGFR-2-based DNA vaccine in mouse models, Genetic Vaccines and Therapy 2009 7.
16. Puja, Debashish B, Shaija S, Lee M E, Targeting Tumor Angiogenesis. Seminars in Oncology 2009 36: S12-S19.
17. Wang Y, Fei D, Vanderlaan M, Song A. Biological activity of bevacizumab, a humanized anti-VEGF antibody in vitro. Angiogenesis 2004; 7: 335-45.
18. Jocelyn H, Sam D, Nick P, Susan D C, Lillian H, Michelle R et al., VEGF-Trap: a VEGF blocker with potent antitumor effects. Proc Natl Acad Sci USA 2002; 99: 11393-8.
19. Fukasawa M, Korc M., Vascular endothelial growth factor-trap suppresses tumorigenicity of multiple pancreatic cancer cell lines. Clin Cancer Res 2004; 10: 3327-32.

20. Jianzhong H, Jason S F, Anna S, Angela K, Akiko Y, Kimberly W M et al., Regression of established tumors and metastases by potent vascular endothelial growth factor blockade. Proc Natl Acad Sci USA 2003; 100: 7785-90.
21. Hu L, Hofmann J, Holash J, Yancopoulos G D, Sood A K, Jaffe R B. Vascular endothelial growth factor trap combined with paclitaxel strikingly inhibits tumor and ascites, prolonging survival in a human ovarian cancer model. Clin Cancer Res 2005; 11: 6966-71.
22. Reily G J, Miller V A, Vascular Endothelial Growth Factor Trap in Non-Small Cell Lung Cancer. Clin Cancer Res 2007; 13: 4623-4627
23. Juan F, Candelaria G M, Ramon A, Polly S Y L, Timothy J M, Paraskevi M, et al., A mutant oncolytic adenovirus targeting the Rb pathway produces anti-glioma effect in vivo. Oncogene 2000; 19: 2-12.
24. Heise C, Sampson-Johannes A, Williams A, McCormick F, Von Hoff D D, Kim D H: ONYX-015, an E1B gene-attenuated adenovirus, causes tumor-specific cytolysis and antitumoral efficacy that can be augmented by standard chemotherapeutic agents. Nat Med 1997; 3: 639-645.
25. Lee H, Kim J, Lee B, Chang J W, Ahn J, Park J O et al., Oncolytic potential of E1B 55 kDa-deleted YKL-1 recombinant adenovirus: correlation with p53 functional status. Int J Cancer 2000; 88: 454-463.
26. Kim J, Cho J Y, Kim J H, Jung K C, Yun C O: Evaluation of E1B gene-attenuated replicating adenoviruses for cancer gene therapy. Cancer Gene Ther 2002; 9: 725-736.
27. Sauthoff H, Heitner S, Rom W N, Hay J G: Deletion of the adenoviral E1b-19kD gene enhances tumor cell killing of a replicating adenoviral vector. Hum Gene Ther 2000; 11: 379-388.
28. Zhou Z, Zhou R R, Guan H, Bucaba C D, Klenerman E S., E1A gene inhibits angiogenesis in Ewing's sarcoma animal model. Mol Cancer Ther 2003: 2: 1313-1319.
29. Akeo S, Mikito I, Hideharu A, Sachiko Y, Kenya S, Masabumi S., Mapping of the Sites Involved in Ligand Association and Dissociation at the Extracellular Domain of the Kinase Insert Domain-containing Receptor for Vascular Endothelial Growth Factor, The Journal of Biological Chemistry 1998 273: 31283-31288.
30. Florence T. H. Wu, Marianne O. Stefanini, Feilim Mac Gabhann, Aleksander S. Popel, A compartment model of VEGF distribution in humans in the presence of soluble VEGF receptor-1 acting as a ligand trap. PLoS One 2009; 4: 1-36.

1. A recombinant adenovirus with improved angiogenesis inhibition activity comprising: (a) an inverted terminal repeat (ITR) nucleotide sequence of an adenovirus; and (b) a nucleotide sequence coding for a chimeric decoy receptor comprising (i) an extracellular domain of vascular endothelial growth factor receptor 1 (VEGFR-1) and (ii) an extracellular domain of vascular endothelial growth factor receptor 2 (VEGFR-2).

2. The recombinant adenovirus of claim 1, wherein the chimeric decoy receptor comprises at least one extracellular domain of VEGFR-1 selected from a group consisting of a first extracellular domain, a second extracellular domain, a third extracellular domain, a fourth extracellular domain, a fifth extracellular domain, a sixth extracellular domain and a seventh extracellular domain of VEGFR-1 and at least one extracellular domain of VEGFR-2 selected from a group consisting of a first extracellular domain, a second extracellular domain, a third extracellular domain, a fourth extracellular domain, a fifth extracellular domain, a sixth extracellular domain and a seventh extracellular domain of VEGFR-2.

3. The recombinant adenovirus of claim 2, wherein the chimeric decoy receptor comprises: (i) the first extracellular domain of VEGFR-1 and at least one extracellular domain of VEGFR-2 selected from a group consisting of the second extracellular domain, the third extracellular domain, the fourth extracellular domain, the fifth extracellular domain, the sixth extracellular domain and the seventh extracellular domain of VEGFR-2; (ii) the second extracellular domain of VEGFR-1 and at least one extracellular domain of VEGFR-2 selected from a group consisting of the first extracellular domain, the third extracellular domain, the fourth extracellular domain, the fifth extracellular domain, the sixth extracellular domain and the seventh extracellular domain of VEGFR-2; (iii) the third extracellular domain of VEGFR-1 and at least one extracellular domain of VEGFR-2 selected from a group consisting of the first extracellular domain, the second extracellular domain, the fourth extracellular domain, the fifth extracellular domain, the sixth extracellular domain and the seventh extracellular domain of VEGFR-2; (iv) the fourth extracellular domain of VEGFR-1 and at least one extracellular domain of VEGFR-2 selected from a group consisting of the first extracellular domain, the second extracellular domain, the third extracellular domain, the fifth extracellular domain, the sixth extracellular domain and the seventh extracellular domain of VEGFR-2; or (v) the fifth extracellular domain of VEGFR-1 and at least one extracellular domain of VEGFR-2 selected from a group consisting of the first extracellular domain, the second extracellular domain, the third extracellular domain, the fourth extracellular domain, the sixth extracellular domain and the seventh extracellular domain VEGFR-2.

4. The recombinant adenovirus of claim 2, wherein the chimeric decoy receptor comprises: (i) the first extracellular domain of VEGFR-2 and at least one extracellular domain of VEGFR-1 selected from a group consisting of the second extracellular domain, the third extracellular domain, the fourth extracellular domain, the fifth extracellular domain, the sixth extracellular domain and the seventh extracellular domain of VEGFR-1; (ii) the second extracellular domain of VEGFR-2 and at least one extracellular domain of VEGFR-1 selected from a group consisting of the first extracellular domain, the third extracellular domain, the fourth extracellular domain, the fifth extracellular domain, the sixth extracellular domain and the seventh extracellular domain of VEGFR-1; (iii) the third extracellular domain of VEGFR-2 and at least one extracellular domain of VEGFR-1 selected from a group consisting of the first extracellular domain, the second extracellular domain, the fourth extracellular domain, the fifth extracellular domain, the sixth extracellular domain and the seventh extracellular domain of VEGFR-1; (iv) the fourth extracellular domain of VEGFR-2 and at least one extracellular domain of VEGFR-1 selected from a group consisting of the first extracellular domain, the second extracellular domain, the third extracellular domain, the fifth extracellular domain, the sixth extracellular domain and the seventh extracellular domain of VEGFR-1; or (v) the fifth extracellular domain of VEGFR-2 and at least one extracellular domain of VEGFR-1 selected from a group consisting of the first extracellular domain, the second extracellular domain, the third extracellular domain, the fourth extracellular domain, the sixth extracellular domain and the seventh extracellular domain of VEGFR-1.

5. The recombinant adenovirus of claim 3, wherein the chimeric decoy receptor comprises 2-4 extracellular domains.

6. The recombinant adenovirus of claim 4, wherein the chimeric decoy receptor comprises 2-4 extracellular domains.

7. The recombinant adenovirus of claim 5, wherein the chimeric decoy receptor comprises: (i) the first extracellular domain of VEGFR-2, the second extracellular domain of VEGFR-1 and the third extracellular domain of VEGFR-2; (ii) the second extracellular domain of VEGFR-1, the third extracellular domain of VEGFR-2 and the fourth extracellular domain of VEGFR-2; or (iii) the second extracellular domain of VEGFR-1, the third extracellular domain of VEGFR-2, the fourth extracellular domain of VEGFR-2 and the fifth extracellular domain of VEGFR-2.

8. The recombinant adenovirus of claim 6, wherein the chimeric decoy receptor comprises: (i) the second extracellular domain of VEGFR-1, the third extracellular domain of VEGFR-2 and the fourth extracellular domain of VEGFR-1; or (ii) the second extracellular domain of VEGFR-1, the third extracellular domain of VEGFR-2, the fourth extracellular domain of VEGFR-1 and the fifth extracellular domain of VEGFR-1.

9. The recombinant adenovirus of claim 1, wherein the Fc region of immunoglobulin is fused in the chimeric decoy receptor.

10. The recombinant adenovirus of claim 1, wherein the recombinant adenovirus lacks the E3 gene and the nucleotide sequence coding for a chimeric decoy receptor is inserted at the region of the E3 gene.

11. The recombinant adenovirus of claim 1, wherein the recombinant adenovirus comprises an inactivated E1B 19 gene, an inactivated E1B 55 gene or an inactivated E1B 19/E1B 55 gene.

12. The recombinant adenovirus of claim 1, wherein the recombinant adenovirus comprises an active E1A gene.

13. The recombinant adenovirus of claim 1, wherein the recombinant adenovirus has a mutation with the 45th Glu residue of a nucleotide sequence coding for the Rb binding site of the E1A gene substituted with Gly and a mutation with the 121st through 127th amino acids substituted with Gly.

14. An anti-angiogenesis composition comprising: (a) a therapeutically effective amount of the recombinant adenovirus according to claim 1; and (b) a pharmaceutically acceptable carrier.

15. The composition of claim 14, wherein the composition is for prevention or treatment of cancer, diabetic retinopathy, retinopathy of prematurity, corneal transplant rejection, neovascular glaucoma, erythrosis, proliferative retinopathy, psoriasis, hemophilic arthropathy, proliferation of capillaries in atherosclerotic plaques, keloid, wound granulation, vascular adhesion, rheumatoid arthritis, osteoarthritis, autoimmune disease, Crohn's disease, recurrent stricture, atherosclerosis, intestinal tract adhesion, cat scratch disease, ulcer, hepatocirrhosis, glomerulonephritis, diabetic nephropathy, malignant nephrosclerosis, thrombotic microangiopathy, organ transplant rejection, glomerulopathy, diabetes, inflammation or neurodegerative disease.

16. A method for preventing or treating a disease caused by excessive angiogenesis, comprising administering an anti-angiogenesis composition comprising: (a) therapeutically effective amount of the recombinant adenovirus according to claim 1; and (b) a pharmaceutically acceptable carrier to a subject in need thereof.

17. The method of claim 16, wherein the disease caused by excessive angiogenesis is cancer, diabetic retinopathy, retinopathy of prematurity, corneal transplant rejection, neovascular glaucoma, erythrosis, proliferative retinopathy, psoriasis, hemophilic arthropathy, proliferation of capillaries in atherosclerotic plaques, keloid, wound granulation, vascular adhesion, rheumatoid arthritis, osteoarthritis, autoimmune disease, Crohn's disease, recurrent stricture, atherosclerosis, intestinal tract adhesion, cat scratch disease, ulcer, hepatocirrhosis, glomerulonephritis, diabetic nephropathy, malignant nephrosclerosis, thrombotic microangiopathy, organ transplant rejection, glomerulopathy, diabetes, inflammation or neurodegerative disease.


SEQUENCE LISTING (8 sequences)

SEQ ID NO: 1 - DNA, 300 bp, Homo sapiens
  CDS (1)..(300)
ggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagat

SEQ ID NO: 2 - protein, 100 aa, Homo sapiens
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIID

SEQ ID NO: 3 - DNA, 210 bp, Homo sapiens
  CDS (1)..(210)
gtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagat

SEQ ID NO: 4 - protein, 70 aa, Homo sapiens
VVLSPSHGIELSVGEKLVLNCTARTELNVGIDFNWEYPSSKHQHKKLVNRDLKTQSGSEMKKFLSTLTID

SEQ ID NO: 5 - DNA, 378 bp, Homo sapiens
  CDS (1)..(378)
ggtgtaacccggagtgaccaaggattgtacacctgtgcagcatccagtgggctgatgaccaagaagaacagcacatttgtcagggtccatgaaaacctttctgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccg

SEQ ID NO: 6 - protein, 126 aa, Homo sapiens
GVTRSDQGLYTCAASSGLMTKKNSTFVRVHENLSVAFGSGMESLVEATVGERVRIPAKYLGYPPPEIKWYKNGIPLESNHTIKAGHVLTIMEVSERDTGNYTVILTNPISKEKQSHVVSLVVYVPP

SEQ ID NO: 7 - DNA, 690 bp, Homo sapiens
  CDS (1)..(690)
ggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 230 aa, Homo sapiens
GPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK